SEQUENCE LISTING (1 sequence)

SEQ ID NO: 1 - DNA, 8101 bp, Homo sapiens
  gene (1)..(8101): somatostatin receptor 2 (SSTR2) gene
gcagggacagctgggaccagtcgacgtccactggccctctgatggctcctaggactgaatcttggactccaggtgcgggtttacactccctgcgctcattgggaactgcatggagaagcgctatcccctgagccctttttctccctactcttagcctggccctgcgccctgcgcccggggctggcccacggtaaacacagctttgctaacttgtttggctaaggaaatcacagaggtcccggtataagtctgggtcaccccggccgccactccagctgcctagaatatatgggtggaagggaatcgactctgtgaaatcagagggaaaatagcgcttgtccttgccatgagtcttgaggagaccgaaaacgcttaaccttttacgcccccgcaggcgggtcccctctctccccgctccccggctgtctgtaagctctgcctgcggccacccgcaggcgtttcagccggtctcacccctgtccttctgcaggacccgggaggaggggttgggggggcggagcgaagccgctgtgacgtagcgggaggggggcgtggggaaatgtgccgaggggcccgggctggctgggccagtcccagcggcgcagccacccatgcgcgcgcgctcgcaagaccaccagcgcccagagccccagtctgaggcttggcgccgggggtctgcgggcgaggggagctctctacgtgcgaggggctagcgggagccggcacaagagggtcgaggagccaggaaccccaaacgtccggcgccaggcgctagccaagctgctgcgcgccccggcgcccagctggctcggggacagccgctgggtgtcggagaccggagctagcggattgcagcggaaaagcaaaggtgaggggtgtgtgtgtgtgtgtgtgtgtgtgtgtgtgtgtgtgtgtgataagagagatggagggagcgagaagcgcacttgggcacctgtgtgcatctgcgctgatggtggtgtgcccatccgagtgcctgagcttaggtcccggtgcgtgattctccgctcttgtgccttttggggtgattgtagtaggaatgaacgacaacgggtacccttgcctgagtaagggggctgtgggtagagtgtgctggaacggacgtgtcctcgcagcctcatgcccgtgtgcgtggcgtgtgccctttagcccgagatttcaggtagctgcgacgggtgacaacttctctcccagccccctacaaaagagacctggcgcgaggggagcgaggccgtgagatgccagctggggctcctgcgggagcgcacccggagatccgagcctgccagaggcaggcggcgggcgcagagcggagaaagaggggcttctctccctagacgctgaacgatctaggatccgtccccgtcccccacctcgggacagaaaggacagtttgtctaggtttggagagaaaaaaccactgcataggccgtgcccaaaagccgctggccaagtcccccaagcgactgtcttctgcgccccgatgtctctgtcctcagcgccccccccccacacccggcacccctgctgtgcgtttcgatactgggcgtgctggcgccacaatctccgctcttgcctcgtcttcctggaaatggcacagagttctttgggaaacccttgctctgaggatcagcgagttggatggccaggaggaggactttctgtgccagccgggagcaaccggctccgcggtcctgacactcgcccctccatttctcaaccccgtaggccagcaccgccccggcttttcccaggcgctcacgcgccgcggtggccctcaggggcttttgtcaccctgccagtgggggctctcgctctagccgcacagagaccaagccgggttctgcaggccctgagggaggcggggggtgggaagtgaatgcgggaaacatgatggggagaggagaaactgaagctgagtaggatttaggacctcccctgatgtcgggtcgccatcccaacactcatttcttgggctggtaatcacagcccctatgtaaaaggggggcggggggggcaggtgcgtgagaccattctcaccctcctctctacagagcctggacatggttcagaggaaaccaaccactagccatttccagcatctaacaattcttgggctggaaaaacaaagaatgcagaaaacgaaacttccttgtacatttaatttaaccacaattcatctagaattgtctgcctggcattggaatattctttctctgaaacaaaaatgaaacagaagtctctggaagaccttaagcggctgacttctttgttaaataagactccccatgatttaagctcatttcttgcttagaggagccttcccactctcagccggctccccagcctcccacctccaccaccttcaccaagactctgaaccctgtctgttgctaccattaagcaattctgtcctgttgactcaaactccagttaaaatgaccgagttagggctggaaagcaacactcaaccctctctcatactccctgcaccatcatcgttcctagcccaaaagctcttagacaggggctctgccaacccagggggattccgtgttactcagacattggagtgtgaccattcatgttatatagatgggcccctggaaatccccatgataaggtacactctgattgcaggcagcttgaataggattctggctctgtagaattaaaccaactgaccagatggttagaagtgataacgaaactacccaagttaatccagggatactaaccacagtttctgtacagcttctgttttaattgctgccagtctatgcttttttacgcaatgcagacatgaaattccaggtgcctcaaatacttcacaaaatggtcagccacaaagcccagatctcacttcacagacagttgtgtggtagggaaatgagcacagaaggaacgagcaatgcacctggcagttcagaatcaatcagaagcaaaggtgagcaaggatcctcaagtacttgttgctggccaagtctcctttaactgatctgcagtctttccaaggattaagaagtaatcttccatctacacccaggcaccaggaaaaggacctagctcaggggaaatgtgtcagccaagtgaattagtcccactctgctgaacacaccaccctttgaacatctcgcctcttcctagattggcctctttgctgtcctcctgcttcactcttcatatacccaagacccagctcaaacacttctctttggaagcctcctctgagtcccccaggaaaggaaggcattcttaagtccttcatttatctctcgtgcaatgcccaccctatatgagctggcttcctttcctatctccccttttaaattatcacctcctagagggcactggccaagtttgttcatttctacatccctgctgtcagcacaaagaagcctcctctccaggcccccaacccccgtgatattttttgaatggctgtatatcaatcatttaattatgggatgaactattgttttagatcttaagccaagccaatagtgctccaattattttctcagcaaggaagtaacacaggagtcagttgcttcaaaccaaagcccagttatcagccgttcggtctctaggccactgaggagcagaggggatgccttgagacgtgcaaaagacttggggccaggtggcctgtgttcacatcccagctccaccaattatgtgcaagagaatggggtgagctccttaaactctcttaagcctcagtttccacatctctaaaatgggggtaattatccctaccacctaggacagttggggagatcaagggactcgtgagtgtgaatgaattatatcagtactggaagccttctgcttacttctgtgaaagagcttgtgtcccacacctgcttcccgtttttgtccgtaattagaaaatggcaggcaaattctctggagtgttacagcacttgggagcagcatccccttagggactttgggaaagagctcttgaggaagtcaagcattaggtattggaaaacaaaaatagaagaaaaacaaaaaataaactgaagcctacatttcaaaaatgaaagcaaaccagacttttatttttaatactgaagactataaattgtttcaccacgtaggtagatttcaataaatcagagataatgagatggtagaggaaaacatggggggaaacaacttacgaggttcccattatgagcccaacgcaaggctaggcattttcacatatattccatcatttaaccttcatgacgcccccatgtgaagaaataagagtcagaaccattaaggaccaggcatgtggtcacacgggctcagcagtggaacccggtttgttctgcctctagagtctgggttttttccactatggcattttcagaatggaaagactccaaggcagtcagcaagtcagcatagatttcctggtagggaagaggccaggaatgtcagtgtcagacccttctgaggtcaggcgctgaacttctccaagctctgcctttctgcagtttagatcagtcaacttcttaggggtcaaagtatgtgctttttgaagccacagccctccccgacatgtgcgtcagcagatgatggctgaacccaaacccttccctactattggaaaaacaactcaaaaagtctgcacactgatgaggaactctagagcttaatgttgatgtggaaagataatacatttttcaatttaagagtatgtctgagaggctaaaccagaaatgtgtaaatttggtgagactttaaacagcctgtgaccgacgggccaatcttcctcttttccttccagatgtcacactggatccttggcctccagggtccattaaggtgagaataagatctctgggctggctggaactagcctaagactgaaaagcagccatggacatggcggatgagccactcaatggaagccacacatggctatccattccatttgacctcaatggctctgtggtgtcaaccaacacctcaaaccagacagagccgtactatgacctgacaagcaatgcagtcctcacattcatctattttgtggtctgcatcattgggttgtgtggcaacacacttgtcatttatgtcatcctccgctatgccaagatgaagaccatcaccaacatttacatcctcaacctggccatcgcagatgagctcttcatgctgggtctgcctttcttggctatgcaggtggctctggtccactggccctttggcaaggccatttgccgggtggtcatgactgtggatggcatcaatcagttcaccagcatcttctgcctgacagtcatgagcatcgaccgatacctggctgtggtccaccccatcaagtcggccaagtggaggagaccccggacggccaagatgatcaccatggctgtgtggggagtctctctgctggtcatcttgcccatcatgatatatgctgggctccggagcaaccagtgggggagaagcagctgcaccatcaactggccaggtgaatctggggcttggtacacagggttcatcatctacactttcattctggggttcctggtacccctcaccatcatctgtctttgctacctgttcattatcatcaaggtgaagtcctctggaatccgagtgggctcctctaagaggaagaagtctgagaagaaggtcacccgaatggtgtccatcgtggtggctgtcttcatcttctgctggcttcccttctacatattcaacgtttcttccgtctccatggccatcagccccaccccagcccttaaaggcatgtttgactttgtggtggtcctcacctatgctaacagctgtgccaaccctatcctatatgccttcttgtctgacaacttcaagaagagcttccagaatgtcctctgcttggtcaaggtgagcggcacagatgatggggagcggagtgacagtaagcaggacaaatcccggctgaatgagaccacggagacccagaggaccctcctcaatggagacctccaaaccagtatctgaactgcttggggggtgggaaagaaccaagccatgctctgtctactggcaatgggctccctacccacactggcttcctgcctcccacccctcacacctggcttctagaatagaggattgctcagcatgagtccaattcagagaacggtgtttgagtcagcttgtctgattgaatgataatgtgctaaattgattacctcccccttaaagcgaacactgaaatgcaggtagacaattcaaagtctggagaagagggatcatgcctggatatgatctttagaaacaacaaaaatagaaaaaaataagtatctgtgtgtttgtgtattgaaaactcaatatgtaatcttgtgtttttatatgtatacttgtatattcctatttattctctgtataggcattacctacgttcctgtgtttacatacacaagtagcaaattcgagtatgcatagtgtagatggacatttgccacaacacactgcccgcagaaatggacttaccgtgaagccaataaagttcaagcttcagggatctctcttgcacgggccttgccaaggcccaggagggacttgggcagtatgttcatgtggtcatatgtttttgtaaaaaattgtgaaagtaagatatgtttgtattgtttttcttaaagaggaacctcgtataagcttcaagcctcacaaaccttctagcctctgcccttggggatttgcttcattaatttcaggcaagtgaggtcaatgtaagaagggaaagggagaagatatttgaagaaccagaatgtaaattcatgtgtttccacttctcagatatagtcagagaattattcatttgcccaaaaggacttaagtggttgtggtcatccatcattgtatttatcaagacaaagccaactttgttataagattgcatttttttcttttcaaattgctttagtttttcttagggagctatgagggggaaaaatcactaacatgaaaggcaaaaaatggactatgattcctgtggggaaacaatttcattctctccatcgtgaaaataagtgaataagagtgaagcaaaattacacctttatgagaaaccataaaattgtttttatttttcaggccagacatagcttcctaatgaaagaaaatggaaatgtaattcgacgactcctcaaaggggactttagaggacttcatacaaagctgggcattaagaaaaccacaatgcatggccgggcgtggtggcttacacctgtaatcccagcactttgggaggccgaggtgggtggatcacccgaggtcaggagttcgagaccagcctggccaacatggtgaaaccccatcactactaaaaatatgtaaattagtcgggcgtggtgtcacgtgcctgtaatcctagctgctcgggaggctgaggcaggagaatcacttgaacttgggaggtggaggttgcagtaagctgagattgtgccactgcactctagcctgagcaacaagagcaaaactcagtctcaaaaaaaaaaaaaaaaaaaaaaaaaaaaaaaaaaaaggaaaaccacaatgcgtactaaagaccagaagacattgttcacaaaacaaaagcaccctcacctgccaatgaatatgcagcgtgcagggggtgtggtgtgagtgttggtggggccccacctctcggagatactgctgtgctgccttcctcactgactgtatacatagtaactgtcatatctttaatgccatggactcactgagccgctctgcaaggactattgtagacaggcacttcacaccataaagtggcatttttttcgttccccaaactgacatttacaagcgataagaaaagagacaatatccatttcattgactgatcattttctagagtatgaagaaatacacacctgggtgtctgcaaggatgtcatcatctttgggtttcatctgagagcatcactcagcatctcacacatagatgttaccatatttttaaatgagctttcctcatccggctccctaagcaagcgctgttggccggtgggagtgactaagtgctccacctgtgggtgtccttcttaatgtgctgcttttgttctgtataaattcacaccacctca

(19) United States
(12) Patent Application Publication
(10) Pub. No.: US 2014/0288011 A1
(43) Pub. Date: Sep. 25, 2014

(54) GENETIC ASSOCIATION

(71) Applicant: David John Grainger, Royston (GB)

(72) Inventor: David John Grainger, Royston (GB)

(73) Assignee: FUNXIONAL THERAPEUTICS LIMITED, Cambridge (GB)

(21) Appl. No.: 14/001,505

(22) PCT Filed: Feb. 28, 2012
(86) PCT No.: PCT/GB2012/050446

§ 371 Date: Mar. 24, 2014

(30) Foreign Application Priority Data
Feb. 28, 2011 (GB) 1103407.1

(51) Int. Cl. C12Q 1/68

(52) U.S. Cl. CPC C12Q 1/6883
USPC 514/21.6; 435/6.11; 506/9; 506/2

(57) ABSTRACT

This invention is directed in part to methods, assays and/or kits for identifying an individual who has an autoimmune disease (such as rheumatoid arthritis), or who has an altered risk for having or developing the autoimmune disease. The methods in one aspect comprise determining the presence or absence of a nucleic acid variant within the somatostatin receptor type 2 (sstr2) gene in the individual's nucleic acids, wherein the presence of the nucleic acid variant is correlated with having the autoimmune disease or the altered risk. The nucleic acid variant may, for example, be a single nucleotide polymorphism (SNP).

The invention relates to methods for identifying individuals who have an autoimmune disease, or who have an altered risk for having or developing the autoimmune disease, and related kits, assays and uses.

Autoimmune diseases arise when an individual's immune system elicits a response against his/her own cells and tissues. Examples of autoimmune diseases include rheumatoid arthritis (RA), systemic lupus erythematosus (SLE), multiple sclerosis (MS), Crohn's disease, Grave's disease, mysethenia gravis, scleroderma, Sjorgren's syndrome, Churg-Strauss Syndrome, Hashimoto's thyroiditis, Addison's disease, autoimmune haemolytic anaemia, idiopathic thrombocytopenic purpura, pernicious anaemia, pemphigus vulgaris, vitiligo, and autoimmune type I diabetes mellitus (T1DM).

Autoimmune diseases have been classified into systemic and organ-specific autoimmune disorders, depending on the principal clinical or pathologic features of the disease. Systemic autoimmune diseases are usually associated with auto-antibodies to antigens that are not organ- or tissue-specific, and include the diseases RA and SLE. Organ-specific (or local) autoimmune diseases affect a specific organ or tissue, and include the diseases T1DM and coeliac disease.

The pathological mechanisms causing most autoimmune diseases have not yet been elucidated. Susceptibility to autoimmune diseases is associated with multiple risk factors. Nevertheless, a genetic contribution to some autoimmune diseases has been established on the basis of a generally higher disease rate in monozygotic (identical) twins compared with dizygotic (non-identical) twins or other family members. Autoimmunity is understood to develop when genetically predisposed individuals encounter (poorly understood) environmental agents that trigger the disease. Environmentally induced epigenetic changes, such as altered DNA methylation patterns which affect gene expression, are considered to play a role in the pathology of some autoimmune diseases.

RA is estimated to affect up to 3% of the population worldwide (reviewed in Goronzy & Weyand, 2010, Arthritis Research & Therapy 11: 249; Hewagama & Richardson, 2009, J. Autoimmun. 33: 3). The disease is characterised by chronic synovial inflammation and progressive destruction of the joint architecture. Although RA has been extensively studied, the etiology and pathogenesis of the disease remain incompletely understood. However, irreversible joint destruction can be prevented by intervention at the early stages of the disease, so early diagnosis and treatment of RA is beneficial. Currently, diagnosis of RA is difficult and some symptoms of RA resemble those of other diseases. The use of immunological tests (such as measurement of the levels of rheumatoid factor [RF] or anti-citrullinated peptide antibodies [ACPAs]) is complicated and on their own may not be sufficiently sensitive or indicative of RA.

Factors that may increase the risk for RA include the sex, age and genetics of an individual. Familial and twin studies suggest that overall there is a greater than 50% genetic contribution to RA. Genes which have been identified as associated with RA include protein tyrosine phosphatase, non-receptor type 22 (lymphoid) (PTPN22; chromosome location: 1p13), peptidylarginine deiminase 4 (PADI4; chromosome location: 1p36), Tumour necrosis factor receptor superfamily member 1B (TNFRSF1B; chromosome location: 1p36), signal transducer and activator of transcription 4 (STAT4; chromosome location: 2q32), programmed cell death 1 (PDCD1; chromosome location: 2q37), solute carrier family 22 (organic cation/ergothioneine transporter), member 4 (SLC22A4; chromosome location: 5q31), major histocompatibility complex, class II, DR beta 1 (HLA-DRB1; chromosome location: 6p21) and runt-related transcription factor 1 (RUNX1; chromosome location: 21q22) (see Goronzy & Weyand, 2010, supra, and Hewagama & Richardson, 2009, supra, and references cited in both). Thus far, the contribution of human leukocyte antigen (HLA) genes at 6p21 shows the strongest linkage to RA, with a familial risk factor of only about 30%. Overall, the genetic polymorphisms identified to date are deemed to be neither necessary nor sufficient for disease development as they are too infrequent and their associated risk is low. However, it is considered that the respective pathways in which the genes or their products are involved are likely to be of importance in rendering an individual susceptible to RA development (Goronzy & Weyand, 2010, supra).

SLE is characterised by the production of antinuclear antibodies, the generation of circulating immune complexes, and the activation of the complement system. SLE is notable for unpredictable exacerbations and remissions. The disease may typically affect an individual's joints, skin, kidney, brain, serosa, lung, heart, and gastrointestinal tract. As with RA, a genetic contribution to SLE is known. Recent reviews of SLE genetics (see for example, Hewagama & Richardson, 2009, supra, and references cited therein) indicate that there are more than 20 loci containing SLE-associated genes. These include PTPN22, FCGR2A, FCGR3A, IL10, C1Q, STAT4, CTLA4, PDCD1, PXK, IL21, C2, C4, TNFA, TNFB, IRF5, IFNA, IFNB, MBL, IFNG, ITGAM, MAN2B1, C3 and MECP2, located on chromosomes 1, 2, 3, 4, 6, 7, 9, 10, 12, 16, 19 and X. Of these, PTPN2 and STAT4 are also associated with susceptibility to RA.

T1DM is characterised by insulin deficiency, caused by beta cell destruction. It is a further example of an autoimmune disease with genetic and environmental components. In a study based on the population of Sardinia, common genetic elements at chromosome regions 6q26, 10q21.2, 20p12.3 and 22q11.22 were shown to contribute to a higher prevalence of T1DM (and MS) (see Hewagama & Richardson, 2009, supra, and references cited therein). The major locus determining T1DM familial aggregation has been shown to be an HLA region on chromosome 6p21. Other loci associated with T1DM include INS, PTPN22, PTPN2, IL2RA, CTLA4 and IFIH1 located on chromosomes 1, 2, 10, 11 and 18. Of these, PTPN2 is also associated with susceptibility to RA and SLE.

MS is a chronic inflammatory neurodegenerative autoimmune disease which similarly is understood to be caused by a combination of genetic and environmental factors. Thus, an HLA gene cluster positioned at chromosome 6p21.3 has been shown to be associated with MS by both candidate gene association and whole genome linkage analysis (see Hewagama & Richardson, 2009, supra, and references cited therein). Other loci associated with MS include IL2RA, IL7R, TNFA, IL1RA, APOE, CD58 and CD24 located on chromosomes 1, 2, 5, 6 and 10. These genes include cytokines and their receptors which may drive the inflammatory process in MS.

Treatment of autoimmune diseases is currently immunosuppressive, anti-inflammatory or merely palliative. The severity of certain diseases can be manipulated by changes in diet and/or use of steroidal or NSAID drugs. Currently used immunotherapies—such as TNF-α antagonists (for example, etanercept), B-cell depleting agents (for example, rituximab) and/or anti-IL-6 receptor antibodies (for example, tocilizumab) for treating RA and other autoimmune diseases—carry a risk of certain adverse effects such as susceptibility to infection.

Although clinically distinct, autoimmune diseases do have similarities in their pathogenesis. The diseases typically involve the production of cytokines and chemokines, important protein mediators that play a key role in regulating the inflammatory response and in the induction, regulation and amplification of autoimmune diseases. It is likely therefore, as noted above for RA, SLE and MS, that autoimmune diseases may share common genetic factors. Common and/or disease-specific genetic factors may assist in early and better diagnosis of the diseases. Also, it has been found that polymorphisms in genes encoding proteins involved in regulating the immune response and inflammation at least partially correlate with differing responses of autoimmune disease subjects to treatment. Elucidation of further genetic factors associated with autoimmune diseases is therefore highly desirable.

According to the present invention, there is provided in one aspect a method for identifying an individual who has an autoimmune disease, or who has an altered risk for having or developing the autoimmune disease, comprising determining the presence or absence of a nucleic acid variant within the somatostatin receptor type 2 (sstr2) gene in the individual's nucleic acids, wherein the presence of the nucleic acid variant is correlated with having the autoimmune disease or the altered risk.

The sstr2 gene which is located on human chromosome 17 encodes the SSTR2 receptor which has been identified as the target receptor for peptide and aminolactam broad-spectrum chemokine inhibitors (BSCIs), as described for example in WO2010/097600 and publications cited therein. The association between nucleic acid variants within the sstr2 gene and autoimmune disease, as demonstrated here for the first time, is unexpected and presents the first strong genetic risk factor for autoimmune diseases such as RA found on chromosome 17. Applications and uses of the association are described herein.

In the method of the invention, determining may be performed on a biological sample from the individual, for example on blood, sputum, saliva, mucosal scraping or tissue biopsy.

The nucleic acid variant may be a single nucleotide polymorphism (SNP).

The autoimmune disease may be one or more of the group consisting of rheumatoid arthritis (RA), systemic lupus erythematosus (SLE), multiple sclerosis (MS), Crohn's disease, Grave's disease, mysethenia gravis, scleroderma, Sjorgren's syndrome, Churg-Strauss Syndrome, Hashimoto's thyroiditis, Addison's disease, autoimmune haemolytic anaemia, idiopathic thrombocytopenic purpura, pernicious anaemia, pemphigus vulgaris, vitiligo, and autoimmune type I diabetes mellitus (T1DM).

The autoimmune disease may in particular be a systemic disease, for example RA.

According to the method of the invention, the presence of the nucleic acid variant may be not correlated with an altered risk for osteoarthritis. In other words, the nucleic acid variant is specific for an autoimmune disease.

The altered risk may be an increased risk.

The sstr2 gene may be defined by the nucleotide sequence of SEQ ID NO: 1.

Nucleotide sequences which have at least 95%, for example at least 96%, 97%, 98% or 99%, sequence identity to SEQ ID NO: 1, for example calculated over the entire length of SEQ ID NO: 1, are also encompassed by the term “sstr2 gene”.

Sequence identity between nucleotide sequences can be determined by comparing an alignment of the sequences. When an equivalent position in the compared sequences is occupied by the same base, then the molecules are identical at that position. Scoring an alignment as a percentage of identity is a function of the number of identical bases at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W 2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for nucleotide sequence comparisons (“DNA Molecule” option) may be Gap Extend penalty: 0.5, Gap Open penalty: 10.0, Matrix: DNAfull.

The nucleic acid variant may be within a non-coding region of the sstr2 gene.

The nucleic acid variant may be a SNP selected from the group consisting of: rs12936744, rs11077670, rs728291, rs998571 and optionally rs2236752.

The nucleic acid variant in particular may be a SNP genotype selected from the group consisting of: rs12936744 (such as the G/G polymorphism or haplotype), rs11077670 (such as the G/G polymorphism or haplotype), rs728291 (such as the NA polymorphism or haplotype), rs998571 (such as the NA polymorphism or haplotype) and optionally rs2236752 (such as the G/G polymorphism haplotype). The haplotypes indicated here are strongly associated with having an autoimmune disease such as RA or an increased risk for having or developing same, as demonstrated in Example 1.

Each of the SNPs or SNP genotypes may be assessed according to the invention.

According to the method, determining may additionally or alternatively comprise assessing the presence or absence of a genetic marker that is in linkage disequilibrium with the nucleic acid variant.

Determining may comprise one or more of the group consisting of: nucleic acid amplification (for example, PCR), primer extension, restriction endonuclease digestion, sequencing, oligonucleotide hybridisation (such as SNP-specific oligonucleotide hybridisation), and a DNAse protection assay. Further means of determining are described below.

The individual may be a white Caucasian, based on the population group in Example 1.

The method may further comprise a step of treating the individual based on the results of the method.

In another aspect of the invention there is provided a method for assessing the severity, stage or progress of an autoimmune disease (such as RA) in an individual, comprising the steps of:

(i) detecting the presence or absence of a nucleic acid variant within the somatostatin receptor type 2 (sstr2) gene in the individual's nucleic acids, wherein said variant is indicative of the autoimmune disease; and
(ii) measuring or monitoring the levels of IGF-1 in the individual.

The method may further comprise the step of detecting the presence or absence of one or more further markers for the autoimmune disease (see below).

In a further aspect there is provided a method of monitoring the treatment of an individual with an autoimmune disease (such as RA), comprising the steps of:

(i) assessing the severity, stage or progress of the autoimmune disease using the method defined herein; and
(ii) administering a treatment agent to the individual.

An additional aspect of the invention provides a method for screening an agent for the treatment of an autoimmune disease (such as RA), comprising the steps of assessing the severity or progress of the autoimmune disease in an individual using the method defined herein before and after administering the agent to the individual, thereby determining whether or not the agent is suitable for the treatment of the autoimmune disease.

The agent may an anti-inflammatory compound such as a BSCI (as defined in WO2010/097600).

Another aspect of the invention is a method for identifying whether or not an individual would benefit from treatment with an anti-inflammatory compound (such as a BSCI), comprising determining the presence or absence of a nucleic acid variant within the somatostatin receptor type 2 (sstr2) gene in the individual's nucleic acids.

Further provided is an assay for identifying an individual who has an autoimmune disease (such as RA), or who has an altered risk for having or developing the autoimmune disease (such as RA), wherein the assay comprises means for determining the presence or absence of a nucleic acid variant within the sstr2 gene in the individual's nucleic acids.

The means for determining the presence or absence of the nucleic acid variant may, for example, be one or more sstr2 allele-specific primers and/or sstr2-specific probes (such as oligonucleotide probes, PNA probes and/or other artificial probes). Further suitable means are described below.

The assay may, for example, be a nucleic acid microarray (such as a DNA microarray).

Additionally provided according to the invention is a kit for assessing whether or not an individual will respond to treatment of a disease involving the sstr2 gene, comprising means for detecting the presence of at least one nucleic acid variant in the sstr2 gene. The means may be one or more allele-specific primers and/or sstr2-specific probes (such as oligonucleotide probes, PNA probes and/or other artificial probes). Further suitable means are described below.

Also provided is a kit comprising:

(i) means for determining the presence or absence of a nucleic acid variant within the somatostatin receptor type 2 (sstr2) gene in an individual's nucleic acids; and
(ii) instructions for identifying whether or not the individual has an autoimmune disease (such as RA), or has an altered risk for having or developing the autoimmune disease (such as RA), based on the presence or absence of a nucleic acid variant determined in step (i).

The means for determining the presence or absence of the nucleic acid variant may as defined herein.

The invention further encompasses the use of the kit as defined above for identifying whether or not an individual has an autoimmune disease (such as RA), or has an altered risk for having or developing the autoimmune disease (such as RA).

Additionally provided is a method for treating an individual with an autoimmune disease (such as RA), comprising the step of:

(i) detecting whether or not the individual has a nucleic acid variant within the sstr2 gene in the individual's nucleic acids, wherein the variant is indicative of the presence of, or risk of developing, the autoimmune disease; and
(ii) if yes, administering an anti-inflammatory compound (such as a BSCI) to the individual.

Also provided is a computer program product for use in determining a predisposition for an autoimmune disease (such as RA) in an individual, the computer program product having a computer readable medium encoded with a program code which comprises a first computer code for receiving, at a host computer, information indicating the presence or absence of a nucleic acid variant within the sstr2 gene in the individual's nucleic acids, and a second computer code for determining a predisposition for an autoimmune disease in the individual, wherein a predisposition for an autoimmune disease is predicted if the nucleic acid variant within the sstr2 gene is present.

The one or more further markers mentioned above may, for example, be a citrullinated peptide (a marker for RA), which may be detected using anti-citrullinated peptide antibodies (“ACPAs”). The detection of ACPAs may employ immunoassays based on detecting the binding with an antigen known to be recognised by these antibodies, for example a natural citrullinated peptide or a synthetic citrullinated peptide (such as peptide A [pepA] or peptide B [pepB]). Binding of the ACPAs to the antigen can be detected for example by a labelled secondary antibody such as a fluorescently-labelled secondary antibody. Immuno-assays may be either competitive or noncompetitive. Non-competitive immunoassays are assays in which the amount of captured analyte is directly measured. In competitive assays, the amount of analyte present in the sample is measured indirectly by measuring the amount of an added (exogenous) analyte displaced (or competed away) from a capture agent by the analyte present in the sample. Suitable immunological methods include enzyme-linked immunosorbent assays (ELISA), immunoblotting, immunospotting (such as line immunoassays or LIA), radioimmunoassays (RIA), fluid or gel precipitation reactions, immunodiffusion (single or double), agglutination assays, Immunoelectrophoresis, time-resolved immunofluorometric assay (TRIFMA), Western blots, liposome immunoassays, complement-fixation assays, immunoradiometric assays, fluorescent immunoassays, protein A immunoassays or immunoPCR. The presence of ACPAs can be detected either in vivo or in vitro, but suitably detection of is performed in vitro on a biological sample obtained from the subject.

Another example of the one or more further markers is Rheumatoid factor (RF), a marker for RA and/or SLE. RF is an autoantibody which can bind to the Fc portion of other antibodies. It is not normally found in healthy individuals, but has been associated with several autoimmune diseases such as RA and SLE, as well as other diseases. Even though RF is not specific for RA, and not all patients diagnosed with RA are RF positive, it is a common marker used to assist diagnosis of RA.

Different methods for determining the presence of RF are known, including agglutination tests (such as a Waaler-Rose assay or sheep cell agglutination test, or a test with latex particles coated with human IgG), rate or laser nephelometry, ELISA, capillary precipitation, and an immunoassay.

Alternatively, the one or more further markers may be for known genetic factors associated with the autoimmune disease. As elaborated in the introduction section above, these markers may be for any one or more of the group of genes consisting of:

### (1) for RA: PTPN22, PADI4, TNFRSF1B, STAT4, PDCD1, SLC22A4, HLA-DRB1 and RUNX1;

### (2) for SLE: PTPN22, FCGR2A, FCGR3A, IL10, C1Q, STAT4, CTLA4, PDCD1, PXK, IL21, C2, C4, TNFA, TNFB, IRF5, IFNA, IFNB, MBL, IFNG, ITGAM, MAN2B1, C3 and MECP2;

(3) for T1DM: the HLA region on chromosome 6p21, INS, PTPN22, PTPN2, IL2RA, CTLA4 and IFIH1; and
(4) for MS: the HLA gene cluster positioned at chromosome 6p21.3, IL2RA, IL7R, TNFA, IL1RA, APOE, CD58 and CD24.

The step of determining the presence of a nucleic acid variant in the sstr2 gene according to various methods of the present invention may be carried out in vivo or in vitro. In one aspect, detection of nucleic acid variants in the sstr2 gene is performed in vitro on a biological sample obtained from the individual.

A nucleic acid comprising a sequence of interest may be obtained from a biological sample comprising DNA (e.g. gDNA or cDNA) or RNA (e.g. mRNA). If required, concentration and/or isolation of the nucleic acid from the sample can be done by any method known in the art or using commercial kits (such as the QIAamp DNA Blood Kit from Qiagen (Hilden, Germany) for the isolation of nucleic acids from blood samples, the ‘High pure PCR Template Preparation Kit’ (Roche Diagnostics, Basel, Switzerland) or the DNA purification kits (PureGene, Gentra, Minneapolis, US). Other well-known procedures for the isolation of DNA or RNA from a biological sample are also available (see for example Sambrook et al., Molecular Cloning: a Laboratory Manual, Cold Spring Harbor Laboratory Press, 1989, Cold Spring Harbor, US; and Ausubel et al., Current Protocols in Molecular Biology, 2003, John Wiley & Sons). When the quantity of nucleic acid is low or insufficient for the assessment, the nucleic acid of interest may be amplified. Amplification may be accomplished by methods known in the art, including, for example, the polymerase chain reaction (PCR), ligase chain reaction (LCR), nucleic acid sequence-based amplification (NASBA), strand displacement amplification, rolling circle amplification, T7-polymerase amplification, and reverse transcription polymerase chain reaction (RT-PCR).

The methods of the present invention optionally comprise the steps of isolating nucleic acids from the sample and/or an amplification step.

Numerous means and methods for detecting single nucleotide differences in nucleic acid sequences are known in the art and can be used in the present invention. Examples include: allele-specific PCR methods such as intercalating dye, FRET primers, and Alphascreen™; primer extension methods such as ARMS (amplification refractory mutation system), kinetic or real-time PCR, SNPstream™, Genetic Bit Analysis™ (GBA), multiplex minisequencing, SnaPshot™, Pyrosequencing™, MassEXTEND™, MassArray™, the MALDI mass spectrometry-based “GOOD” assay, microarray minisequencing, APEX (arrayed primer extension), sequence specific priming (SSP), microarray primer extension, Tag arrays, coded microspheres, template-directed incorporation (TDI), fluorescence polarization; oligonucleotide ligation methods such as colorimetric OLA (oligonucleotide ligation assay), sequence-coded OLA, microarray ligation, ligase chain reaction, padlock probes, and rolling circle amplification; hybridisation methods such as reverse dot blot, line probe assay (LiPA), GeneChip™ microarrays, dynamic allele-specific hybridization (DASH), peptide nucleic acid (PNA) and locked nucleic acid (LNA) probes, TaqMan™ (5′ nuclease assay), and molecular beacons; and endonuclease cleavage methods such as restriction site analysis (RFLP) and Invader™ assay.

The detection of the presence or absence of a nucleic acid variant may for example be determined by DNA or RNA hybridization, sequencing, PCR, primer extension, multiplex ligation-dependent probe amplification (MLPA), oligonucleotide ligation assay (OLA) or restriction site analysis.

In a further aspect of the invention there is provided a method of diagnosing whether a subject has, or is at risk of, an autoimmune disease (such as RA), in which the method comprises determining the sstr2 haplotype of the subject, for example by detecting one or more or all SNPs as defined herein which are distinctive of the sstr2 gene.

Further provided is an autoimmune disease (such as RA) diagnosis kit which comprises means for determining the sstr2 haplotype of an individual.

Additionally provided is the use of the autoimmune disease diagnosis kit as defined above to determine the sstr2 haplotype of an individual.

Also provided is the use of one or more probes capable of binding specifically to a region of nucleic acid which includes an SNP distinctive of the sstr2 gene in an individual for the diagnosis of an autoimmune disease (such as RA) in the individual.

Additionally provided is the use of a primer capable of amplifying sstr2 nucleic acid which includes an SNP distinctive of an sstr2 allele for the diagnosis of an autoimmune disease (such as RA).

In another aspect of the invention there is provided a method of running a diagnostic business, comprising:

(i) determining the sstr2 haplotype of an individual using a method of the invention as defined herein; and
(ii) notifying the individual or a healthcare provider of the result.

In a further aspect there is provided a method of conducting a bioinformatics business, comprising:

(i) determining the sstr2 haplotype of a plurality of different individuals using a method of the invention as defined herein; and
(ii) generating a database comprising information recording the sstr2 haplotype of the different individuals.

Further features related to the above methods, kit and uses are as elaborated elsewhere herein.

SNPs are defined herein according to their reference SNP identity number (“rs . . . ”) assigned by the dbSNP database of the National Center for Biotechnology Information (NCBI). The dbSNP database is incorporated into the NCBI's Entrez system.

As used herein, the term “gene” refers not only to the coding sequence but also to all sequences that are part of that gene, including the introns and exons, the regulatory regions such the promoter region and possible other regulatory sequences, such as 5′UTR, 3′UTR or sequences further up- or downstream.

The term “haplotype” as used herein refers to a set of associated alleles. The term “haplotype” may thus refer to specific nucleic acid variants (such as SNP polymorphisms) within the somatostatin receptor type 2 (sstr2) gene. For example, an individual may have an haplotype of “G/G” at the rs12936744 SNP, of “G/G” at the rs11077670 SNP, “NA” at the rs728291 SNP, “A/A” at the rs998571 SNP, and/or “G/G” at the rs2236752 SNP, all which are shown herein to be associated with RA (see Example 1).

Further features and particular non-limiting embodiments of the present invention will now be described below with reference to the following drawings, in which:

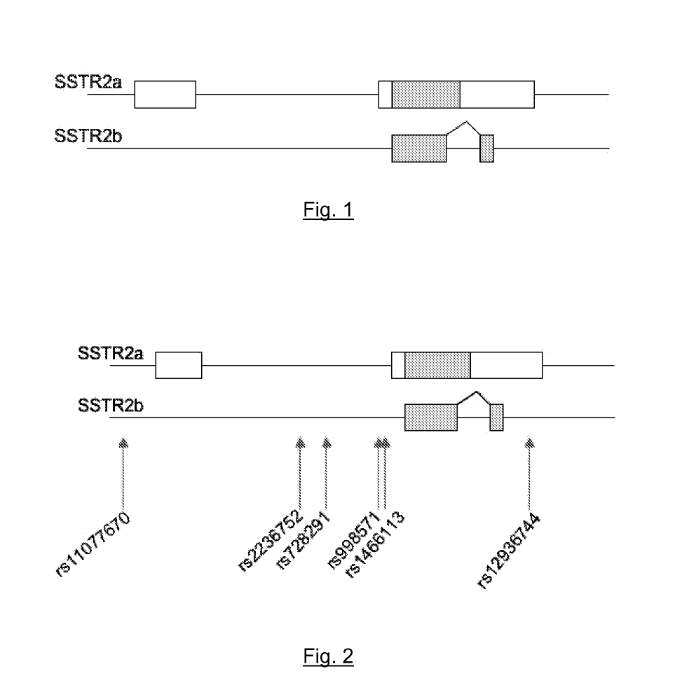

FIG. 1 is a diagrammatic representation of the exon structure of the sstr2 gene. The darker regions represent the coding frame; and

FIG. 2 is a diagrammatic representation of the location of the six selected tag SNPs with respect to the intron/exon structure and coding sequence of the sstr2 gene. The SNP locations shown in FIG. 2 are approximate.

### EXAMPLE 1

This example examined genetic variation at the sstr2 locus, which encodes the type 2 somatostatin receptor, and analysed whether this variation was associated with rheumatoid arthritis (RA) and/or osteoarthritis (OA).

### Study Design

The study was a conventional cross-sectional genetic association study in a cohort of unrelated subjects. Multiple single nucleotide polymorphisms (SNPs) were used to tag as much of the genetic variability at the target locus, and both the individual SNPs and a best estimate of the haplotype constructed from those SNPs were tested for association with the presence of RA.

Patients were recruited without relying on the population prevalence of RA or OA to determine the number of cases and controls in the cohort under analysis. This design is more powerful than a conventional case control cohort design, minimising the selection bias inherent in separately defined recruitment criteria for cases and controls.

### Methods

### Patient Population

The analysis was performed on stored DNA from the MaGiCAD cohort (see www.magicad.org.uk for details), described in Mosedale et al. (2005; Atherosclerosis 183:268-74, which reference is incorporated herein in its entirety). This cohort consists of 1,234 randomly selected patients presenting at a single centre (Papworth Hospital, Cambridgeshire, UK) for coronary angiography as a result of symptoms consistent with coronary heart disease, together with 100 partners of the recruited patients.

All patients arriving at the hospital for angiography were eligible (except for patients who have previously undergone a heart transplant), and recruited patients were selected randomly from the angiography lists. Random selection was confirmed by comparison of more than 20 demographic variables between the recruited patients and all patients on the angiography lists.

The recruited subjects were 68% male, with an average age of 61.2 years.

DNA was prepared from whole peripheral blood taken from the angiography catheter sheath in the femoral artery (except for the partners, where blood was obtained by conventional venepuncture). Only a subset (74.8%) of the subjects in MaGiCAD consented to provide DNA samples for analysis, and this subset was randomly distributed with respect to RA and OA status.

The subjects recruited into MaGiCAD are exceptionally well characterized. More than five hundred separate parameters are recorded for each subject, including detailed demographic and anthropomorphic characteristics, medical history, family history of disease and current phenotype. In addition, a large number of hormones, cytokines, metabolites and genetic data have already been collected and recorded in the central database, allowing extensive investigation of intermediate phenotypes in genetic association studies.

The presence of RA or OA was defined in the study population by the current prescription of drugs for the treatment of RA or OA.

### SNP Selection

### Resources Used:

Gene, transcript and protein sequence information: Ensembl (EMBL-EBI and Welcome Trust Sanger Institute, UK)
Identification of SNPs and SNP frequencies: Genecards (Weizmann Institute of Science, US), dbSNP (NCBI, US), HapMap (NCBI, US), Applied Biosystems™ (US)
Selection of tag SNPs: HapMap, Tagger (Broad Institute, US)
Published SNP associations: PubMed (NCBI, US).

The sstr2 gene is situated on Chromosome 17q24. There are two published locations of the sstr2 gene; originally the chromosomal location was noted as 68,672,755-68,679,655 bp on the forward strand, but it is more recently noted as 71,161,160-71,168,060 bp on the forward strand (Ensembl gene ID: ENSG00000180616; SEQ ID NO: 1).

The gene yields a transcript which is alternatively spliced and subsequently translated to yield two highly homologous protein products designated sstr2a and sstr2b, which differ only in the C-terminal tail (see FIG. 1).

Ensembl was used to relate the location of the two alternative transcriptional start sites, the protein coding sequence and splice sites, as well as the location of published SNPs. The old sequence locations recorded in Ensembl version (v54) match the SNP locations recorded in Genecards and HapMap.

TABLE 1
Compiled listing of SNPs located in the region of the
sstr2 gene locus detected in the Central European
(CEU; white Caucasian) HapMap population.

| SNP ID | Chr. 17 | | | AB | |
|---|---|---|---|---|---|
| (dbSNP) | Position | Change | MAF | kit? | References |
| rs34739008 | 68670823 | C/T | <0.014 | Yes | |
| rs35790859 | 68671009 | C/T | 0.026 | Yes | |
| rs11077670 | 68671381 | G/A | ≧0.079 | Yes | Sutton (2006) |
| | | | | | Sutton et al. |
| | | | | | (2009; Diabetes |
| | | | | | 58: 1457-1462) |
| rs2881097 | 68671414 | C/G | 0.118 | Yes | |
| rs34901075 | 68671415 | C/T | 0.329 | No | |
| rs35841232 | 68671913 | G/T | <0.014 | No | |
| rs35351585 | 68672138 | C/T | 0.014 | Yes | |
| rs2236750 | 68674202 | G/A | 0.225 | Yes | |
| rs2236752 | 68674792 | T/A | 0.255 | Yes | |
| rs714925 | 68675264 | A/G | 0.417 | Yes | Sutton (2006) |
| | | | | | Sutton et al. |
| | | | | | (2009; supra) |
| rs728291 | 68675383 | C/A | 0.383 | Yes | |
| rs2236754 | 68675676 | A/G | 0.225 | Yes | |
| rs1037261 | 68675971 | G/A | 0.441 | Yes | |
| rs998571 | 68676887 | A/G | 0.400 | Yes | Torrisani (2001) |
| | | | | | Filopanti |
| | | | | | M (2005) |
| | | | | | Canzian (2005) |
| | | | | | Sutton (2006) |
| | | | | | Wagner (2006) |
| | | | | | Sutton et al. |
| | | | | | (2009; supra) |
| rs1466113 | 68676913 | C/G | 0.441 | Yes | Torrisani (2001) |
| | | | | | Filopanti (2005) |
| | | | | | Canzian (2005) |
| | | | | | Fox et al. (2007; |
| | | | | | BMC Medical |
| | | | | | Genetics 8 |
| | | | | | [Suppl I]: S18) |
| rs7220818 | 68678296 | A/G | 0.233 | No | Sutton (2006) |
| | | | | | Sutton et al. |
| | | | | | (2009; supra) |
| rs7210080 | 68678697 | T/C | 0.225 | Yes | |
| rs7210093 | 68678728 | T/C | 0.211 | No | |
| rs7224362 | 68679136 | A/G | 0.171 | No | |
| rs12936744 | 68679350 | G/T | 0.035 | Yes | |
| rs11655730 | 68672725 | G/T | 0.130 | No | † |

In addition to the SNP ID, Table 1 shows the location on chromosome 17 (using Ensembl v54 numbering), together with the nucleotide change. None of the listed SNPs are associated with a nonsynonymous change in the coding region of the gene. The minimum allele frequency (MAF) in the HapMap CEU population is also shown. The availability of a pre-made kit from Applied Biosystems™ is also indicated (‘AB kit?’), together with any published references referring to the particular SNP. † This SNP was not present in the databases when the tag SNP set was selected, but was subsequently discovered and added to the database during our gene analysis.

From the list in Table 1, HapMap and Tagger were used to select a set of tag SNPs to tag the sstr2 gene. A tag set of SNPs represents a selected subset of SNPs that, due to their frequency and linkage characteristics, together capture the maximum proportion of local genetic variability in the smallest number of SNPs. In addition, SNPs that have been reported in publications (so are more ‘validated’ as actual SNPs, and have frequency data for a reasonable population size), as well as SNPs with a genotyping assay kit available from Applied Biosystems™ were prioritised for inclusion in the tag set.

Using the selection criteria MAF>5%, r²>0.8 (consistent with many publications of similar association study designs), the tag set of SNPs shown in Table 2 was selected. Note that the Tagger program selects rs12936744 as one of the tag SNPs, and this SNP has varying published frequencies around the 5% MAF cutoff, including some less than 5%.

TABLE 2
Location and genotype assay kit information for the
six SNPs selected to form the tag set for the sstr2 locus.

| | | | Applied | |
|---|---|---|---|---|
| | Location in | | Biosystems ™ | |
| SNP ID | sstr2 Gene | Change | Kit ID | Kit type |
| rs11077670 | 5' upstream in both | G/A | C_2167495_10 | Func- |
| | sstr2a and 2b | | | tionally |
| | | | | tested |
| rs2236752 | Intron 1-2 in sstr2a (5' | T/A | C_2167499_1_ | Validated |
| | upstream in sstr2b) | | | |
| rs728291 | Intron 1-2 in sstr2a (5' | C/A | C_2167501_1_ | Validated |
| | upstream in sstr2b) | | | |
| rs998571 | Intron 1-2 in sstr2a. | A/G | C_2167503_10 | Validated |
| | Just upstream of | | | |
| | transcription start site | | | |
| | for sstr2b and start | | | |
| | of protein coding | | | |
| | for both sstr2a and | | | |
| | 2b. Likely synonymous | | | |
| | with A-167G and | | | |
| | A-83G in various | | | |
| | publications | | | |
| rs1466113 | Intron 1-2 in sstr2a. | C/G | C_2167504_10 | Validated |
| | Just upstream of | | | |
| | transcription start site | | | |
| | for sstr2b and start | | | |
| | of protein coding | | | |
| | for both sstr2a and | | | |
| | 2b. Likely synonymous | | | |
| | with C-57G in various | | | |
| | publications | | | |
| rs12936744 | Exon 2 of sstr2a. 3' | G/T | C_32134408_10 | Func- |
| | downstream of the end | | | tionally |
| | of the protein coding | | | tested |
| | region for both sstr2a | | | |
| | and 2b. | | | |

### SNP Genotyping

DNA samples from the MaGiCAD cohort, stored at Medical Solutions Ltd (Nottingham, UK), were provided by TCP Innovations Ltd (Cambridge, UK), the commercial sponsor of the MaGiCAD cohort. All available DNA samples were genotyped for the SNPs using TaqMan assays listed in Table 2, supplied by Applied Biosystems™. Genotyping was carried out by Medical Solutions Ltd (formerly MRC Geneservice) using an ABI Prism 7900HT system and SDS scoring software.

### Results

### Data Processing

The results files from Medical Solutions Ltd were combined, and the genotypes read into a master data file in SPSS format. Where the MaGiCAD database contained a red flag associated with the DNA sample, the genotypes were removed from the master data file prior to further analysis. The reasons for the red flags are listed in Table 3 below. After exclusions, genotypes (including assay failures) were obtained for 987 unique individuals in the MaGiCAD cohort.

TABLE 3
Data from some sample was eliminated
prior to analysis because of
red flags in the MaGiCAD database.

| Sample study | Reason for |
|---|---|
| number | exclusion |
| 609 | Mis-numbered |
| 610 | Mis-numbered |
| 286 | ‘Duplicated’ sample |
| 322 | ‘Duplicated’ sample |
| 425 | ‘Duplicated’ sample |
| 474 | ‘Duplicated’ sample |
| 475 | ‘Duplicated’ sample |
| 514 | ‘Duplicated’ sample |
| 2593 | ‘Duplicated’ sample |
| 2670 | ‘Duplicated’ sample |
| 2688 | ‘Duplicated’ sample |
| 2691 | ‘Duplicated’ sample |
| 2733 | ‘Duplicated’ sample |
| 5161 | ‘Duplicated’ sample |
| 379 | Repeat of patient 546 |
| 501 | Repeat of patient 599 |
| 5081 | Repeat of patient 559 |
| 5099 | Repeat of patient 477 |

There were concerns that two samples may have been misnumbered, while two DNA samples were prepared (in error) for a number of patients, so the data from the second sample was eliminated. Four patients were recruited twice into the MaGiCAD cohort, because the attended Papworth Hospital twice for angiography in the recruitment period, and the study protocol did not exclude repeat recruitment if randomly selected. The data was retained from the first visit of the same individual.

The rates at which the separate genotypes can be called in each assay are assessed as part of the internal quality control process at Medical Solutions Ltd. A cut-off of 90% pass rate was applied. Where some plates have a call rate above 90% and others below 90% for the same assay, the plates that failed quality control were re-assayed. Where all assay plates fail and the operator considers the failure was due to the properties of the assay itself, no repeat was performed and the data was considered unreliable. Five of the six SNPs assayed here met the quality control criteria for the call rate. However, for SNP rs1466113 the low call rate was considered to be due to failure of the assay, and the data therefore considered unreliable.

The four subjects recruited twice into the cohort provide a simple quality control check, since their genotype should be the same on each independent determination. For three of the six SNPs, all four pairs of calls from the same individual were concordant, and for two of the remaining three, there was a single error (see Table 4). The remaining SNP (rs1466113), however, gave random genotypes and this together with the low call rate resulted in this SNP being dropped from the tag set used in the haplotype analysis.

TABLE 4
Genotypes at the six tag SNPs independently determined on two occasions for
four individuals recruited twice into the MaGiCAD cohort.

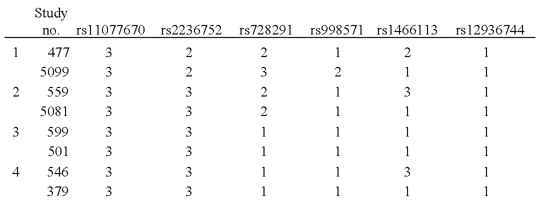

Discordant genotype calls are highlighted.

In addition to the samples from the repeat patients, which were assayed blind by the contract laboratory, Medical Solutions Ltd also deliberately introduced duplicate samples into each run as an additional quality control. All five of these were concordant for all the remaining five tag SNPs (with rs1466113 excluded; Table 5).

TABLE 5
Genotypes at the five remaining tag SNPs (with rs1466113 excluded)
determined in duplicate during genotyping at Medical Solutions Ltd.

| | Sample | | | | | |
|---|---|---|---|---|---|---|
| | code | rs11077670 | rs2236752 | rs728291 | rs998571 | rs12936744 |
| 1 | GS1a | 3 | 3 | 2 | 2 | 1 |
| | GS1b | 3 | 3 | 2 | 2 | 1 |
| 2 | GS2a | 3 | 1 | 3 | 1 | 1 |
| | GS2b | 3 | 1 | 3 | 1 | 1 |
| 3 | GS3a | 3 | 1 | 3 | 1 | 1 |
| | GS3b | 3 | 1 | 3 | 1 | 1 |
| 4 | GS6a | 2 | 3 | 2 | 2 | 2 |
| | GS6b | 2 | 3 | 2 | 2 | 2 |
| 5 | GS7a | 3 | 1 | 3 | 1 | 1 |
| | GS7b | 3 | 1 | 3 | Missing | 1 |

There were no discordant genotype calls.

### Genotype Frequencies

The genotype frequencies for the entire genotyped population are tabulated in Table 6.

TABLE 6
Genotype frequencies for the five remaining tag SNPs for
the entire genotyped population, expressed as
total numbers and as a percentage of the called genotypes.

| rs11077670 | A/A | A/G | G/G | Unscored |
|---|---|---|---|---|
| No. | 7 | 165 | 812 | 3 |
| % called | 0.7 | 16.8 | 82.5 | n/a |
| Call rate: 99.7% | | | | |
| rs2236752 | A/A | A/T | T/T | Unscored |
| No. | 59 | 363 | 560 | 5 |
| % called | 6.0 | 37.0 | 57.0 | n/a |
| Call rate: 99.5% | | | | |
| rs728291 | A/A | A/C | C/C | Unscored |
| No. | 138 | 449 | 383 | 17 |
| % called | 14.2 | 46.2 | 39.5 | n/a |
| Call rate: 98.3% | | | | |
| rs998571 | A/A | A/G | G/G | Unscored |
| No. | 426 | 445 | 109 | 7 |
| % called | 43.5 | 45.4 | 11.1 | n/a |
| Call rate: 99.3% | | | | |
| rs12936744 | G/G | G/T | T/T | Unscored |
| No. | 813 | 163 | 7 | 4 |
| % called | 82.7 | 16.6 | 0.7 | n/a |
| Call rate: 99.6% | | | | |

The genotype distribution for each SNP was tested for Hardy-Weinberg Equilibrium (HWE), initially in the whole population, using an on-line calculator tool at http://www.genes.org.uk/software/hardy-weinberg.shtml. A significant deviation from HWE indicates a potential selection issue in the recruitment of the cohort, or a loss of individuals of a particular genotype from the population (for example, due to premature death of those individuals). All the tag SNPs used here were in HWE in this cohort as a whole: rs11077670 χ²=0.19, p>0.05; rs2236752 χ²=0.00, p>0.05; rs728291 χ²=0.12, p>0.05; rs998571 χ²=0.20, p>0.05; rs12936744 χ²=0.14, p>0.05.

The minimum allele frequencies were also calculated for the whole population (Table 7). The minimum allele frequencies are similar to those published.

TABLE 7
Calculated minimum allele frequencies (MAFs)
for the five tag SNPs in the entire genotyped
population from the MaGiCAD cohort.

| | Minimum | Minimum allele |
|---|---|---|
| SNP | allele | frequency |
| rs11077670 | A | 0.09 |
| rs2236752 | A | 0.24 |
| rs728291 | A | 0.37 |
| rs998571 | G | 0.34 |
| rs12936744 | T | 0.09 |

The MAFs are consistent with previous reports for populations dominated by white Caucasians (see Table 1).

Prior to testing the association with RA and OA, genotypes from individuals with an ethnicity other than Caucasian were excluded from analysis to reduce the possibility of population stratification (an artefact that can result in false positive associations, due to variations in the prevalence of a disease in different ethnic groups associating with the substantially greater differences in genotype distributions between ethnic compared to within an ethnic group). As a result, a further 14 genotypes were eliminated from the analysis.

Association with RA and OA in the Whole Genotyped Population

The control group alone (that is, the subjects without RA or OA) was tested for deviations from HWE. The association between genotype distribution at each of the tag SNPs in turn with the presence of RA and OA was evaluated by a Chi-squared test of the crosstabulation, as shown below in Table 8. Note that the total number (“no.”) of samples in each test may differ due to the different number of assay fails in each genotype assay.

TABLE 8
Chi-squared test of association at each
SNP (parts 8.1-8.5) with the presence
or absence (“No”) of RA and/or OA
8.1 rs12936744

| | GG | GT | TT |
|---|---|---|---|
| No | 313 | 57 | 5 |
| RA | 196 | 20 | 0 |
| OA | 78 | 13 | 1 |

| | |
|---|---|
| RA versus none: | Chi-squared 7.435, df = 2; p = 0.0243 |
| OA versus none: | Chi-squared 0.107, df = 2; p = 0.9481 |

8.2 rs11077670

| | AA | AG | GG |
|---|---|---|---|
| No | 5 | 59 | 313 |
| RA | 0 | 13 | 196 |
| OA | 1 | 13 | 77 |

| | |
|---|---|
| RA versus none: | Chi-squared 14.29, df = 2; p = 0.0008 |
| OA versus none: | Chi-squared 0.141, df = 2; p = 0.9322 |

8.3 rs2236752

| | AA | AG | GG |
|---|---|---|---|
| No | 25 | 130 | 222 |
| RA | 11 | 61 | 146 |
| OA | 8 | 37 | 47 |

| | |
|---|---|
| RA versus none: | Chi-squared 3.853, df = 2; p = 0.1457 |
| OA versus none: | Chi-squared 1.915, df = 2; p = 0.3838 |

8.4 rs728291

| | AA | AC | CC |
|---|---|---|---|
| No | 56 | 172 | 142 |
| RA | 97 | 63 | 59 |
| OA | 13 | 40 | 37 |

| | |
|---|---|
| RA versus none: | Chi-squared 61.12, df = 2; p < 0.0001 |
| OA versus none: | Chi-squared 0.227, df = 2; p = 0.8925 |

8.5 rs998571

| | AA | AG | GG |
|---|---|---|---|
| No | 170 | 161 | 45 |
| RA | 133 | 63 | 16 |
| OA | 44 | 42 | 6 |

| | |
|---|---|
| RA versus none: | Chi-squared 16.74, df = 2; p = 0.0002 |
| OA versus none: | Chi-squared 2.259, df = 2; p = 0.3232 |

A summary of the results from Table 8 is given in Table 9.

TABLE 9
Summary of results from Table 8

| | RA MAF | RA | OA MAF | OA |
|---|---|---|---|---|
| SNP | odds | p value | odds | p value |
| rs12936744 | <0.43 | 0.02 | 0.84 | 0.95 |
| rs11077670 | <0.43 | 0.0008 | 0.84 | 0.93 |
| rs2236752 | 0.77 | 0.15 | 1.39 | 0.38 |
| rs728291 | 2.16 | <0.0001 | 0.91 | 0.89 |
| rs998571 | 0.60 | 0.0002 | 0.57 | 0.32 |

The data present in Tables 8 and 9 show that variation at the rs12936744, rs11077670, rs728291 and rs998571 SNPs are associated in a statistically significantly manner with RA, but not OA, in the whole genotyped population.

### CONCLUSIONS

Genetic variation at the sstr2 locus is associated with RA, but not OA, in the MaGiCAD cohort. Genetic variation at the rs12936744, rs11077670, rs728291 and rs998571 SNPs in particular, and less so the rs2236752 SNP, have been found to be associated with RA. These SNPs, as shown in FIG. 2, span the non-coding regions in the sstr2 locus. It is suggested therefore that other SNPs within the sstr2 locus may also be associated with RA.

Although sstr2 is one of several genes which may be involved in the RA pathway, the data in this example in combination with the known role of SSTR2 receptor strongly suggest that the SSTR2 receptor may have a pathogenic role in the development of RA and potentially other autoimmune diseases.

### Sequence Information

SEQ ID NO: 1 - Human sstr2 gene (5′-3′)
Source: Ensembl ID ENSG00000180616
(Chromosome:GRCh37:17:71160560:71168660:1)
GCAGGGACAGCTGGGACCAGTCGACGTCCACTGGCCCTCTGATGGCTCCT
AGGACTGAATCTTGGACTCCAGGTGCGGGTTTACACTCCCTGCGCTCATT
GGGAACTGCATGGAGAAGCGCTATCCCCTGAGCCCTTTTTCTCCCTACTC
TTAGCCTGGCCCTGCGCCCTGCGCCCGGGGCTGGCCCACGGTAAACACAG
CTTTGCTAACTTGTTTGGCTAAGGAAATCACAGAGGTCCCGGTATAAGTC
TGGGTCACCCCGGCCGCCACTCCAGCTGCCTAGAATATATGGGTGGAAGG
GAATCGACTCTGTGAAATCAGAGGGAAAATAGCGCTTGTCCTTGCCATGA
GTCTTGAGGAGACCGAAAACGCTTAACCTTTTACGCCCCCGCAGGCGGGT
CCCCTCTCTCCCCGCTCCCCGGCTGTCTGTAAGCTCTGCCTGCGGCCACC
CGCAGGCGTTTCAGCCGGTCTCACCCCTGTCCTTCTGCAGGACCCGGGAG
GAGGGGTTGGGGGGGCGGAGCGAAGCCGCTGTGACGTAGCGGGAGGGGGG
CGTGGGGAAATGTGCCGAGGGGCCCGGGCTGGCTGGGCCAGTCCCAGCGG
CGCAGCCACCCATGCGCGCGCGCTCGCAAGACCACCAGCGCCCAGAGCCC
CAGTCTGAGGCTTGGCGCCGGGGGTCTGCGGGCGAGGGGAGCTCTCTACG
TGCGAGGGGCTAGCGGGAGCCGGCACAAGAGGGTCGAGGAGCCAGGAACC
CCAAACGTCCGGCGCCAGGCGCTAGCCAAGCTGCTGCGCGCCCCGGCGCC
CAGCTGGCTCGGGGACAGCCGCTGGGTGTCGGAGACCGGAGCTAGCGGAT
TGCAGCGGAAAAGCAAAGGTGAGGGGTGTGTGTGTGTGTGTGTGTGTGTG
TGTGTGTGTGTGTGTGATAAGAGAGATGGAGGGAGCGAGAAGCGCACTTG
GGCACCTGTGTGCATCTGCGCTGATGGTGGTGTGCCCATCCGAGTGCCTG
AGCTTAGGTCCCGGTGCGTGATTCTCCGCTCTTGTGCCTTTTGGGGTGAT
TGTAGTAGGAATGAACGACAACGGGTACCCTTGCCTGAGTAAGGGGGCTG
TGGGTAGAGTGTGCTGGAACGGACGTGTCCTCGCAGCCTCATGCCCGTGT
GCGTGGCGTGTGCCCTTTAGCCCGAGATTTCAGGTAGCTGCGACGGGTGA
CAACTTCTCTCCCAGCCCCCTACAAAAGAGACCTGGCGCGAGGGGAGCGA
GGCCGTGAGATGCCAGCTGGGGCTCCTGCGGGAGCGCACCCGGAGATCCG
AGCCTGCCAGAGGCAGGCGGCGGGCGCAGAGCGGAGAAAGAGGGGCTTCT
CTCCCTAGACGCTGAACGATCTAGGATCCGTCCCCGTCCCCCACCTCGGG
ACAGAAAGGACAGTTTGTCTAGGTTTGGAGAGAAAAAACCACTGCATAGG
CCGTGCCCAAAAGCCGCTGGCCAAGTCCCCCAAGCGACTGTCTTCTGCGC
CCCGATGTCTCTGTCCTCAGCGCCCCCCCCCCACACCCGGCACCCCTGCT
GTGCGTTTCGATACTGGGCGTGCTGGCGCCACAATCTCCGCTCTTGCCTC
GTCTTCCTGGAAATGGCACAGAGTTCTTTGGGAAACCCTTGCTCTGAGGA
TCAGCGAGTTGGATGGCCAGGAGGAGGACTTTCTGTGCCAGCCGGGAGCA
ACCGGCTCCGCGGTCCTGACACTCGCCCCTCCATTTCTCAACCCCGTAGG
CCAGCACCGCCCCGGCTTTTCCCAGGCGCTCACGCGCCGCGGTGGCCCTC
AGGGGCTTTTGTCACCCTGCCAGTGGGGGCTCTCGCTCTAGCCGCACAGA
GACCAAGCCGGGTTCTGCAGGCCCTGAGGGAGGCGGGGGGTGGGAAGTGA
ATGCGGGAAACATGATGGGGAGAGGAGAAACTGAAGCTGAGTAGGATTTA
GGACCTCCCCTGATGTCGGGTCGCCATCCCAACACTCATTTCTTGGGCTG
GTAATCACAGCCCCTATGTAAAAGGGGGGCGGGGGGGGCAGGTGCGTGAG
ACCATTCTCACCCTCCTCTCTACAGAGCCTGGACATGGTTCAGAGGAAAC
CAACCACTAGCCATTTCCAGCATCTAACAATTCTTGGGCTGGAAAAACAA
AGAATGCAGAAAACGAAACTTCCTTGTACATTTAATTTAACCACAATTCA
TCTAGAATTGTCTGCCTGGCATTGGAATATTCTTTCTCTGAAACAAAAAT
GAAACAGAAGTCTCTGGAAGACCTTAAGCGGCTGACTTCTTTGTTAAATA
AGACTCCCCATGATTTAAGCTCATTTCTTGCTTAGAGGAGCCTTCCCACT
CTCAGCCGGCTCCCCAGCCTCCCACCTCCACCACCTTCACCAAGACTCTG
AACCCTGTCTGTTGCTACCATTAAGCAATTCTGTCCTGTTGACTCAAACT
CCAGTTAAAATGACCGAGTTAGGGCTGGAAAGCAACACTCAACCCTCTCT
CATACTCCCTGCACCATCATCGTTCCTAGCCCAAAAGCTCTTAGACAGGG
GCTCTGCCAACCCAGGGGGATTCCGTGTTACTCAGACATTGGAGTGTGAC
CATTCATGTTATATAGATGGGCCCCTGGAAATCCCCATGATAAGGTACAC
TCTGATTGCAGGCAGCTTGAATAGGATTCTGGCTCTGTAGAATTAAACCA
ACTGACCAGATGGTTAGAAGTGATAACGAAACTACCCAAGTTAATCCAGG
GATACTAACCACAGTTTCTGTACAGCTTCTGTTTTAATTGCTGCCAGTCT
ATGCTTTTTTACGCAATGCAGACATGAAATTCCAGGTGCCTCAAATACTT
CACAAAATGGTCAGCCACAAAGCCCAGATCTCACTTCACAGACAGTTGTG
TGGTAGGGAAATGAGCACAGAAGGAACGAGCAATGCACCTGGCAGTTCAG
AATCAATCAGAAGCAAAGGTGAGCAAGGATCCTCAAGTACTTGTTGCTGG
CCAAGTCTCCTTTAACTGATCTGCAGTCTTTCCAAGGATTAAGAAGTAAT
CTTCCATCTACACCCAGGCACCAGGAAAAGGACCTAGCTCAGGGGAAATG
TGTCAGCCAAGTGAATTAGTCCCACTCTGCTGAACACACCACCCTTTGAA
CATCTCGCCTCTTCCTAGATTGGCCTCTTTGCTGTCCTCCTGCTTCACTC
TTCATATACCCAAGACCCAGCTCAAACACTTCTCTTTGGAAGCCTCCTCT
GAGTCCCCCAGGAAAGGAAGGCATTCTTAAGTCCTTCATTTATCTCTCGT
GCAATGCCCACCCTATATGAGCTGGCTTCCTTTCCTATCTCCCCTTTTAA
ATTATCACCTCCTAGAGGGCACTGGCCAAGTTTGTTCATTTCTACATCCC
TGCTGTCAGCACAAAGAAGCCTCCTCTCCAGGCCCCCAACCCCCGTGATA
TTTTTTGAATGGCTGTATATCAATCATTTAATTATGGGATGAACTATTGT
TTTAGATCTTAAGCCAAGCCAATAGTGCTCCAATTATTTTCTCAGCAAGG
AAGTAACACAGGAGTCAGTTGCTTCAAACCAAAGCCCAGTTATCAGCCGT
TCGGTCTCTAGGCCACTGAGGAGCAGAGGGGATGCCTTGAGACGTGCAAA
AGACTTGGGGCCAGGTGGCCTGTGTTCACATCCCAGCTCCACCAATTATG
TGCAAGAGAATGGGGTGAGCTCCTTAAACTCTCTTAAGCCTCAGTTTCCA
CATCTCTAAAATGGGGGTAATTATCCCTACCACCTAGGACAGTTGGGGAG
ATCAAGGGACTCGTGAGTGTGAATGAATTATATCAGTACTGGAAGCCTTC
TGCTTACTTCTGTGAAAGAGCTTGTGTCCCACACCTGCTTCCCGTTTTTG
TCCGTAATTAGAAAATGGCAGGCAAATTCTCTGGAGTGTTACAGCACTTG
GGAGCAGCATCCCCTTAGGGACTTTGGGAAAGAGCTCTTGAGGAAGTCAA
GCATTAGGTATTGGAAAACAAAAATAGAAGAAAAACAAAAAATAAACTGA
AGCCTACATTTCAAAAATGAAAGCAAACCAGACTTTTATTTTTAATACTG
AAGACTATAAATTGTTTCACCACGTAGGTAGATTTCAATAAATCAGAGAT
AATGAGATGGTAGAGGAAAACATGGGGGGAAACAACTTACGAGGTTCCCA
TTATGAGCCCAACGCAAGGCTAGGCATTTTCACATATATTCCATCATTTA
ACCTTCATGACGCCCCCATGTGAAGAAATAAGAGTCAGAACCATTAAGGA
CCAGGCATGTGGTCACACGGGCTCAGCAGTGGAACCCGGTTTGTTCTGCC
TCTAGAGTCTGGGTTTTTTCCACTATGGCATTTTCAGAATGGAAAGACTC
CAAGGCAGTCAGCAAGTCAGCATAGATTTCCTGGTAGGGAAGAGGCCAGG
AATGTCAGTGTCAGACCCTTCTGAGGTCAGGCGCTGAACTTCTCCAAGCT
CTGCCTTTCTGCAGTTTAGATCAGTCAACTTCTTAGGGGTCAAAGTATGT
GCTTTTTGAAGCCACAGCCCTCCCCGACATGTGCGTCAGCAGATGATGGC
TGAACCCAAACCCTTCCCTACTATTGGAAAAACAACTCAAAAAGTCTGCA
CACTGATGAGGAACTCTAGAGCTTAATGTTGATGTGGAAAGATAATACAT
TTTTCAATTTAAGAGTATGTCTGAGAGGCTAAACCAGAAATGTGTAAATT
TGGTGAGACTTTAAACAGCCTGTGACCGACGGGCCAATCTTCCTCTTTTC
CTTCCAGATGTCACACTGGATCCTTGGCCTCCAGGGTCCATTAAGGTGAG
AATAAGATCTCTGGGCTGGCTGGAACTAGCCTAAGACTGAAAAGCAGCCA
TGGACATGGCGGATGAGCCACTCAATGGAAGCCACACATGGCTATCCATT
CCATTTGACCTCAATGGCTCTGTGGTGTCAACCAACACCTCAAACCAGAC
AGAGCCGTACTATGACCTGACAAGCAATGCAGTCCTCACATTCATCTATT
TTGTGGTCTGCATCATTGGGTTGTGTGGCAACACACTTGTCATTTATGTC
ATCCTCCGCTATGCCAAGATGAAGACCATCACCAACATTTACATCCTCAA
CCTGGCCATCGCAGATGAGCTCTTCATGCTGGGTCTGCCTTTCTTGGCTA
TGCAGGTGGCTCTGGTCCACTGGCCCTTTGGCAAGGCCATTTGCCGGGTG
GTCATGACTGTGGATGGCATCAATCAGTTCACCAGCATCTTCTGCCTGAC
AGTCATGAGCATCGACCGATACCTGGCTGTGGTCCACCCCATCAAGTCGG
CCAAGTGGAGGAGACCCCGGACGGCCAAGATGATCACCATGGCTGTGTGG
GGAGTCTCTCTGCTGGTCATCTTGCCCATCATGATATATGCTGGGCTCCG
GAGCAACCAGTGGGGGAGAAGCAGCTGCACCATCAACTGGCCAGGTGAAT
CTGGGGCTTGGTACACAGGGTTCATCATCTACACTTTCATTCTGGGGTTC
CTGGTACCCCTCACCATCATCTGTCTTTGCTACCTGTTCATTATCATCAA
GGTGAAGTCCTCTGGAATCCGAGTGGGCTCCTCTAAGAGGAAGAAGTCTG
AGAAGAAGGTCACCCGAATGGTGTCCATCGTGGTGGCTGTCTTCATCTTC
TGCTGGCTTCCCTTCTACATATTCAACGTTTCTTCCGTCTCCATGGCCAT
CAGCCCCACCCCAGCCCTTAAAGGCATGTTTGACTTTGTGGTGGTCCTCA
CCTATGCTAACAGCTGTGCCAACCCTATCCTATATGCCTTCTTGTCTGAC
AACTTCAAGAAGAGCTTCCAGAATGTCCTCTGCTTGGTCAAGGTGAGCGG
CACAGATGATGGGGAGCGGAGTGACAGTAAGCAGGACAAATCCCGGCTGA
ATGAGACCACGGAGACCCAGAGGACCCTCCTCAATGGAGACCTCCAAACC
AGTATCTGAACTGCTTGGGGGGTGGGAAAGAACCAAGCCATGCTCTGTCT
ACTGGCAATGGGCTCCCTACCCACACTGGCTTCCTGCCTCCCACCCCTCA
CACCTGGCTTCTAGAATAGAGGATTGCTCAGCATGAGTCCAATTCAGAGA
ACGGTGTTTGAGTCAGCTTGTCTGATTGAATGATAATGTGCTAAATTGAT
TACCTCCCCCTTAAAGCGAACACTGAAATGCAGGTAGACAATTCAAAGTC
TGGAGAAGAGGGATCATGCCTGGATATGATCTTTAGAAACAACAAAAATA
GAAAAAAATAAGTATCTGTGTGTTTGTGTATTGAAAACTCAATATGTAAT
CTTGTGTTTTTATATGTATACTTGTATATTCCTATTTATTCTCTGTATAG
GCATTACCTACGTTCCTGTGTTTACATACACAAGTAGCAAATTCGAGTAT
GCATAGTGTAGATGGACATTTGCCACAACACACTGCCCGCAGAAATGGAC
TTACCGTGAAGCCAATAAAGTTCAAGCTTCAGGGATCTCTCTTGCACGGG
CCTTGCCAAGGCCCAGGAGGGACTTGGGCAGTATGTTCATGTGGTCATAT
GTTTTTGTAAAAAATTGTGAAAGTAAGATATGTTTGTATTGTTTTTCTTA
AAGAGGAACCTCGTATAAGCTTCAAGCCTCACAAACCTTCTAGCCTCTGC
CCTTGGGGATTTGCTTCATTAATTTCAGGCAAGTGAGGTCAATGTAAGAA
GGGAAAGGGAGAAGATATTTGAAGAACCAGAATGTAAATTCATGTGTTTC
CACTTCTCAGATATAGTCAGAGAATTATTCATTTGCCCAAAAGGACTTAA
GTGGTTGTGGTCATCCATCATTGTATTTATCAAGACAAAGCCAACTTTGT
TATAAGATTGCATTTTTTTCTTTTCAAATTGCTTTAGTTTTTCTTAGGGA
GCTATGAGGGGGAAAAATCACTAACATGAAAGGCAAAAAATGGACTATGA
TTCCTGTGGGGAAACAATTTCATTCTCTCCATCGTGAAAATAAGTGAATA
AGAGTGAAGCAAAATTACACCTTTATGAGAAACCATAAAATTGTTTTTAT
TTTTCAGGCCAGACATAGCTTCCTAATGAAAGAAAATGGAAATGTAATTC
GACGACTCCTCAAAGGGGACTTTAGAGGACTTCATACAAAGCTGGGCATT
AAGAAAACCACAATGCATGGCCGGGCGTGGTGGCTTACACCTGTAATCCC
AGCACTTTGGGAGGCCGAGGTGGGTGGATCACCCGAGGTCAGGAGTTCGA
GACCAGCCTGGCCAACATGGTGAAACCCCATCACTACTAAAAATATGTAA
ATTAGTCGGGCGTGGTGTCACGTGCCTGTAATCCTAGCTGCTCGGGAGGC
TGAGGCAGGAGAATCACTTGAACTTGGGAGGTGGAGGTTGCAGTAAGCTG
AGATTGTGCCACTGCACTCTAGCCTGAGCAACAAGAGCAAAACTCAGTCT
CAAAAAAAAAAAAAAAAAAAAAAAAAAAAAAAAAAAAGGAAAACCACAAT
GCGTACTAAAGACCAGAAGACATTGTTCACAAAACAAAAGCACCCTCACC
TGCCAATGAATATGCAGCGTGCAGGGGGTGTGGTGTGAGTGTTGGTGGGG
CCCCACCTCTCGGAGATACTGCTGTGCTGCCTTCCTCACTGACTGTATAC
ATAGTAACTGTCATATCTTTAATGCCATGGACTCACTGAGCCGCTCTGCA
AGGACTATTGTAGACAGGCACTTCACACCATAAAGTGGCATTTTTTTCGT
TCCCCAAACTGACATTTACAAGCGATAAGAAAAGAGACAATATCCATTTC
ATTGACTGATCATTTTCTAGAGTATGAAGAAATACACACCTGGGTGTCTG
CAAGGATGTCATCATCTTTGGGTTTCATCTGAGAGCATCACTCAGCATCT
CACACATAGATGTTACCATATTTTTAAATGAGCTTTCCTCATCCGGCTCC
CTAAGCAAGCGCTGTTGGCCGGTGGGAGTGACTAAGTGCTCCACCTGTGG
GTGTCCTTCTTAATGTGCTGCTTTTGTTCTGTATAAATTCACACCACCTC
A.

Exons of either of the two sstr2 splice variants are underlined in the above sequence. The two splice variant transcripts of sstr2 are also known as SSTR2-201 (Ensembl Transcript ID ENST00000315332) and SSTR2-202 (Ensembl Transcript ID ENST00000357585).

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1. A method for identifying an individual who has an autoimmune disease, or who has an altered risk for having or developing the autoimmune disease, comprising determining the presence or absence of a nucleic acid variant within the somatostatin receptor type 2 (sstr2) gene in the individual's nucleic acids, wherein the presence of the nucleic acid variant is correlated with having the autoimmune disease or the altered risk.

2. The method according to claim 1, in which determining is performed on a biological sample from the individual.

3. The method according to claim 1, in which the nucleic acid variant is a single nucleotide polymorphism (SNP).

4. The method according to claim 1, in which the autoimmune disease is one or more of the group consisting of rheumatoid arthritis (RA), systemic lupus erythematosus (SLE), multiple sclerosis (MS), Crohn's disease, Grave's disease, mysethenia gravis, scleroderma, Sjorgren's syndrome, Churg-Strauss Syndrome, Hashimoto's thyroiditis, Addison's disease, autoimmune haemolytic anaemia, idiopathic thrombocytopenic purpura, pernicious anaemia, pemphigus vulgaris, vitiligo, and autoimmune type I diabetes mellitus (T1DM).

5. (canceled)

6. The method according to claim 1, in which the autoimmune disease is rheumatoid arthritis.

7. The method according to claim 1, in which the presence of the nucleic acid variant is not correlated with an altered risk for osteoarthritis.

8. The method according to claim 1, in which the altered risk is an increased risk.

9. The method according to claim 1, in which the sstr2 gene is defined by the nucleotide sequence of SEQ ID NO: 1.

10. The method according to claim 1, in which the nucleic acid variant is within a non-coding region of the sstr2 gene.

11. The method according to claim 1, in which the nucleic acid variant is a SNP selected from the group consisting of: rs12936744, rs11077670, rs2236752, rs728291 and rs998571.

12. The method according to claim 1, in which the nucleic acid variant is a SNP genotype selected from the group consisting of: rs12936744 (G/G polymorphism), rs11077670 (G/G polymorphism), rs2236752 (G/G polymorphism), rs728291 (NA polymorphism) and rs998571 (A/A polymorphism).

13. The method according to claim 1, in which the nucleic acid variant is a SNP selected from the group consisting of: rs12936744, rs11077670, rs728291 and rs998571.

14. The method according to claim 1, in which the nucleic acid variant is a SNP genotype selected from the group consisting of: rs12936744 (G/G polymorphism), rs11077670 (G/G polymorphism), rs728291 (A/A polymorphism) and rs998571 (NA polymorphism).

15. (canceled)

16. The method according to claim 1, in which determining comprising assessing the presence or absence of a genetic marker that is in linkage disequilibrium with the nucleic acid variant.

17. The method according to claim 1, in which determining comprises one or more of the group consisting of: nucleic acid amplification (for example, PCR), primer extension, restriction endonuclease digestion, sequencing, oligonucleotide hybridisation (such as SNP-specific oligonucleotide hybridisation), and a DNAse protection assay.

18. The method according to claim 2, in which the biological sample is blood, sputum, saliva, mucosal scraping or tissue biopsy.

19. The method according to claim 1, in which the individual is a white Caucasian.

20. The method according to claim 1, further comprising a step of treating the individual based on the results of the method.

21. A method for assessing the severity, stage or progress of an autoimmune disease in an individual, comprising the steps of:
(i) detecting the presence or absence of a nucleic acid variant within the somatostatin receptor type 2 (sstr2) gene in the individual's nucleic acids, wherein said variant is indicative of the autoimmune disease; and
(ii) measuring or monitoring the levels of IGF-1 in the individual.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A method for treating an individual with an autoimmune disease, comprising the step of:
(i) detecting whether or not the individual has a nucleic acid variant within the sstr2 gene in the individual's nucleic acids, wherein the variant is indicative of the presence of, or risk of developing, the autoimmune disease; and
(ii) if yes, administering an anti-inflammatory compound (such as a BSCI) to the individual.

40. (canceled)

41. (canceled)

42. (canceled)
